(19) United States
(12) Patent Application Publication
(10) Pub. No.: US 2018/0263666 A1
(43) Pub. Date: Sep. 20, 2018

(54) MONO TO POLY AXIAL UNIVERSAL PEDICLE SCREW AND METHOD OF USING THE SAME

(71) Applicant: Christopher John Marden CAIN, Denver, CO (US)

(72) Inventor: Christopher John Marden CAIN, Denver, CO (US)

(21) Appl. No.: 15/922,782

(22) Filed: Mar. 15, 2018

Related U.S. Application Data
(60) Provisional application No. 62/471,927, filed on Mar. 15, 2017.

(51) Int. Cl. A61B 17/70

(52) U.S. Cl. CPC A61B 17/7037; A61B 17/8605; A61B 17/7032

(57) ABSTRACT

A universal mono to poly axial pedicle screw and method of securing and manipulating vertebrae of a patient spine using a universal mono to poly axial pedicle screw.

### PRIORITY

The present invention claims the benefit of U.S. Provisional application 62/471,927 filed on Mar. 15, 2017.

### FIELD OF THE INVENTION

The present invention is a novel pedicle screw and method of using the same.

### BACKGROUND OF THE INVENTION

A pedicle screw is used for repairing fractured or deformed bones and specifically for connecting vertebrae to rods used in spinal surgery. Existing pedicle screw systems can have either a fixed or variable connection between the head and shaft of the screw. With a fixed connection, there is no motion between the head and shaft of the screw. Screws with a variable connection and various degrees of mobility can be uniaxial (movement along one axis), uniplanar (movement restricted in one plane) or poly-axial (more than one axis of movement resulting in movement in multiple planes).

FIG. 1 illustrates an existing fixed axis screw. With a fixed axis screw there is no motion between the head and shaft of the screw. Fixed axis screws allow greater ability to manipulate the vertebra into which they are inserted. However, the fixed axis increases the difficulty in aligning fixation points over multiple levels of fixation. The fixed axis screw also presents a challenge in contouring the rod connecting fixation points to ensure good fit and a secure connection between the screw head and the rod.

FIG. 2 illustrates existing screws with a variable connection and various degrees of mobility that can be uniaxial (movement along one axis), uniplanar (movement restricted in one plane) or poly axial (more than one axis of movement resulting in movement in multiple planes). With a uniaxial connection, the head screw can rotate within the head but without the ability for angulation between the head and shaft of the screw. A uniaxial connection offers the ability to manipulate the spine as is possible with a fixed angle screw. A uniplanar connection allows the head to rotate and angle but in only one plane. The third connection can be poly axial, which allows angulation between the head and the shaft of the screw enabling movement in multiple planes.

Poly axial screws make it easier to align and connect fixation points over multiple levels. However, due to the mobility of the screw head relative to the shaft of the screw, you lose or at least significantly compromise the ability to manipulate the vertebra directly, either around the rod or prior to placement of the rod. This limits the ability to perform corrective maneuvers useful in restoring alignment in situations of fracture or deformity.

Each of these screw configurations has particular features in relation to enhancing the ability to achieve stabilization and correction of a range of spinal pathologies. However, none provide the ability to manipulate the vertebra into which they are inserted along with the ease of aligning fixation points over multiple levels of fixation. In contrast, the present invention solves the aforementioned limitations and problems of existing screws and provides a single pedicle screw with the ability to manipulate the spine, as is possible with a fixed angle screw, but also provides a variable connection between the head and shaft of the screw providing various degrees of mobility. This transformation occurs as the locking cap for the screw is tightened while maintaining the angle between the screw shaft and the rod. The various degrees of mobility further enable greater flexibility in relation to rod contouring to ensure optimal fit between the locking cap, rod and screw.

### SUMMARY OF THE INVENTION

The present invention provides a pedicle screw with the benefits of a fixed axial screw combined with a poly axial screw for securing and manipulating a spine in a trauma or bone deformity situation. While the primary use of this invention is in spine or vertebra applications, the present invention contemplates applications in other areas of orthopaedics such as external fixation pins for long bone or pelvic injuries or limb deformity correction.

Specifically, the present invention provides a universal mono to poly axial pedicle screw comprising a screw shaft, a screw head, a deformable collar or deformable ridge located below the screw head and around the screw shaft, a U-shaped tulip portion of the screw; wherein the tulip portion comprises a narrow conical end and an open end wherein the narrow end surrounds the screw head and deformable collar or deformable ridge and the open end has interior opposing threads for receiving a threaded locking cap, and wherein the tulip portion of the screw further comprises a cradle with vertical sides, a first concave surface and a second concave surface opposite the first concave surface, wherein the first concave surface surrounds the screw head and the second concave surface receives a securing rod. The open end of the tulip can have removable extension tabs.

The present invention also provides a method of securing and manipulating vertebrae of a patient spine using a universal mono to poly axial pedicle screw of the present invention comprising, inserting the screw in a vertebrae, placing a securing rod on a cradle, manipulating the vertebrae, tightening the locking cap, pulling the tulip over the deformable collar or deformable ridge, further tightening the locking cap, orienting the tulip perpendicular to the securing rod and removing the tulip extension tabs.

### BRIEF DESCRIPTION OF THE DRAWINGS

FIG. 1 illustrates an existing fixed axis pedicle screw.

FIGS. 2A, 2B and 2C illustrate existing uniaxial, uniplanar and poly axial pedicle screws.

FIGS. 3A and 3B illustrate alternate cross sectional side views of the pedicle screw of the present invention.

FIGS. 4A, 4B, 4C and 4D illustrate various levels of insertion and tightening of the locking cap of the present invention.

FIGS. 5A and 5B illustrates initial placement of pedicle screws of the present invention with a securing rod.

FIGS. 6A and 6B illustrate manipulation of the pedicle screws of the present invention.

FIGS. 7A and 7B depict the present invention with the deformable collar.

FIGS. 8A and 8B illustrates the pedicle screw of the present invention with the securing cap.

FIGS. 9A and 9B illustrate two views of the locking cap tightened to the securing rod after the removal of the extension tabs. FIG. 9C shows the pedicle screws inserted into vertebrae.

FIG. 10 illustrates depict the present invention with the deformable ridge.

FIG. 11A is a top view of the coupling screw.

FIGS. 11B and 11C show two views of the tulip extension of the present invention enabling manipulation of the vertebra using the screw.

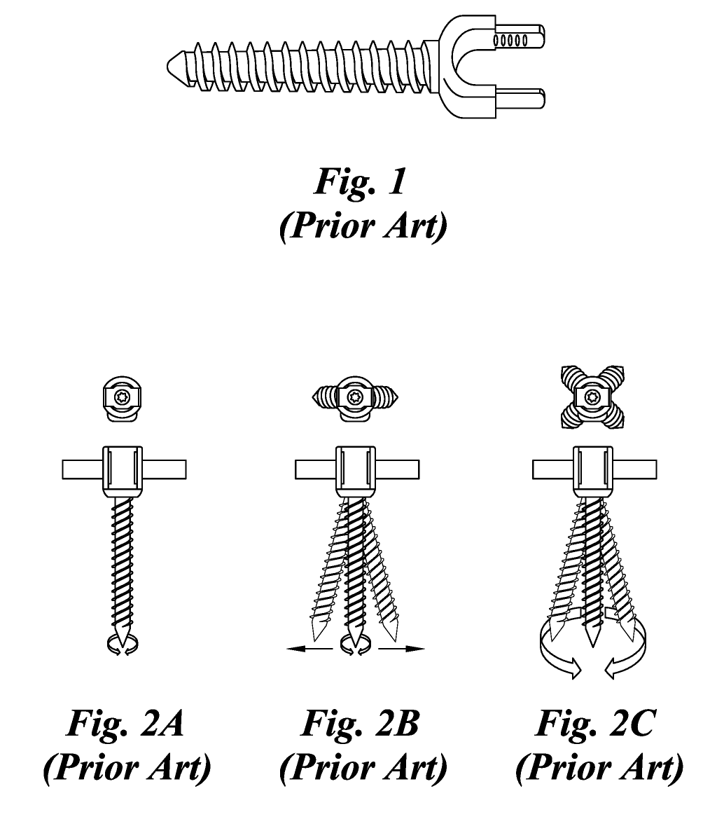

### DETAILED DESCRIPTION OF THE INVENTION

A pedicle screw that has the attributes of a fixed screw in relation to being able to manipulate a vertebra, while preserving the ability for the head of the screw to align itself to the rod, thereby reducing the stress on the screw-bone interface and improving the stability and congruity of the screw-rod connection would be advantageous in a number of clinical situations. One such application is in the management of thoracolumbar spinal trauma. A second application is when performing deformity correction.

The novelty of the present invention relates to the ability to manipulate the spine as is possible with a fixed angle screw but that also provides the benefits of a poly axial screw in relation to rod contouring. Furthermore, the design ensures optimal fit and security between the locking cap, contouring rod and screw as the locking cap is tightened. The transition to a poly axial head during final tightening also reduces the stress that can be placed on the screw bone interface if ideal alignment of the screws or rod contouring is not achieved. Unlike other trauma systems that provide some of these features, this construct also allows the connection of multiple segments, either with similar screw types or with a standard fixed or poly axial screw (as is appropriate for the specified case or clinical situation) that can be utilized above and below this construct.

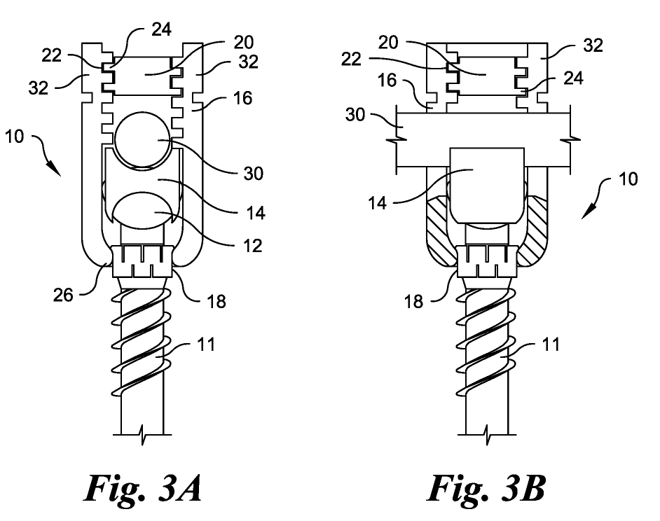

FIGS. 3A and 3B illustrate alternate cross sectional side views of the pedicle screw of the present invention (10). The pedicle screw (10) has a shaft (11) and head (12), a cradle (14), tulip portion of the screw (16) with two opposing vertical sides, a deformable collar (18) surround the screw between the shaft (11) and head (12) and a locking cap (20). The tulip portion of the screw (16) includes opposing interior threads (22) that mate with locking cap threads (24). Extension tabs (32) are located at the upper portion of the tulip (16), which are removable after final tightening of the locking cap (20). Extension tabs (32) can vary in length depending on the application. Generally, the extension tabs (32) can range in length from 10 to 150 mm but can be shorter or longer. The lower limit in length is determined by the parameters of the screw head (12), tulip (16), contouring rod (30) and locking cap (20). There should be a sufficient number of opposing interior threads (22) that allow the use of the suitable rod (30), cradle (14) and locking cap (20) to secure the rod (30) in position. The upper limit in length again depends upon the specific application in order for an open, minimally invasive or percutaneous application.

A rod (30) can be placed through the tulip portion of the screw (16) and between the cradle (14) and locking cap (20). In a preferred embodiment, the tulip portion of the screw (16) has a narrow conical end (26) that initially surrounds the deformable collar (18). The locking cap (20) can be tightened down to compress the rod (30) into the cradle (14) thereby enabling rod contouring to ensure optimal fit between the cap, rod and screw. In a preferred embodiment, the cradle is cylindrical in shape in order to sit securely within the tulip portion of the screw (16). But any shape cradle can be used provided it fits securely in the tulip portion of the screw (16) and has opposing surfaces to mate securely with the screw head (12) and securing rod (30).

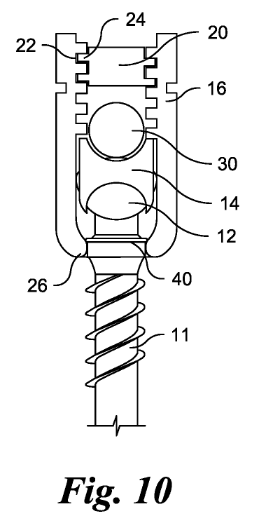

In another preferred embodiment, in lieu of a deformable collar, a deformable ridge (40) on the screw can be used as shown in FIG. 10. The deformable ridge (40) on the screw (11) should give way as the locking cap (20) is tightened and the tulip (16) pulled up over the screw (11). The dimensions of the deformable ridge (40), envisaged to be made of the same material as the screw shaft, can be varied in relation to its width and depth in order to adjust the resistance offered, and force required to allow the transformation from a mono axial to poly axial configuration. The use of a compressible material between the head of the screw and base of the tulip may also deliver the same utility. The compressible material can be any suitable biocompatible non-toxic material with sufficient density to provide the necessary resistance when tightening and manipulating the screw and rod. Such materials can be silicone or rubber or Teflon for example.

While the use of a deformable collar or a deformable ridge are preferred methods for providing the desired features, specifically a fixed or uniaxial screw which becomes poly axial as the cap is tightened, other options exist to achieve a similar outcome. For example, a blocking sleeve, pin or tab incorporated into the head of the screw and or the tulip that can be removed as the cap is being tightened and which doing so would allow realignment of the tulip to facilitate coupling to the rod, while maintaining pressure from the rod on the screw head thereby maintaining the angulation of the screw itself. The use of a compressible material, between the head of the screw and the base of the tulip that deforms under a defined degree of compression or modifications to the cradle itself can also provide this functionality.

In all configurations it is envisaged that final tightening of the cap onto the rod would elevate the cradle of the screw and lock the position of the screw shaft relative to the tulip so that the position of the tulip relative to the screw shaft would be maintained even if the cap were released. This would enable the application of compression or distraction between fixation points without loss of the correction achieved by the initial manipulation of the vertebra.

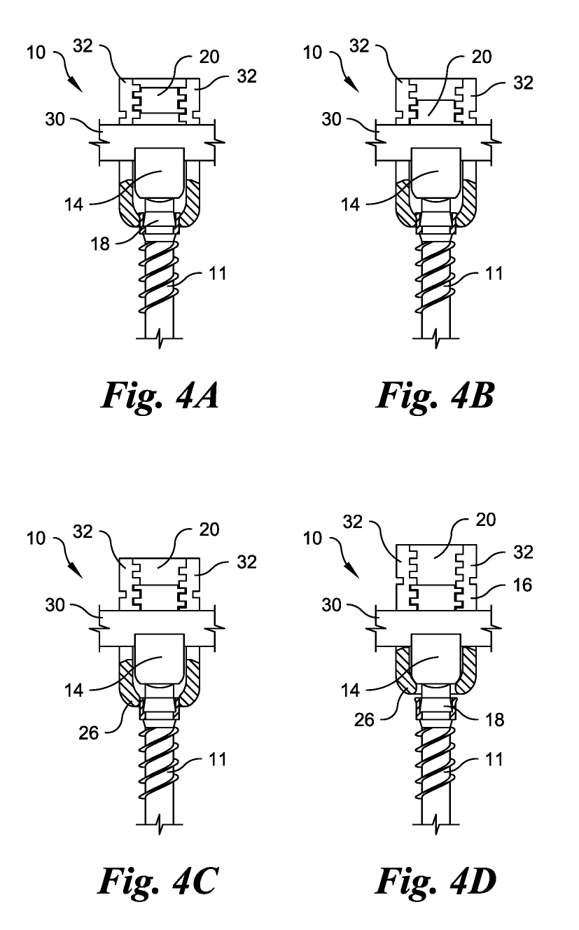

FIGS. 4A, 4B, 4C and 4D illustrate various levels of insertion of the pedicle screw (10) of the present invention into a vertebra and tightening of the locking cap (20). In FIG. 4A, the screw is inserted into bone and the rod (30) is resting on the cradle (14). FIG. 4B illustrates tightening of the locking cap (20) down on the cradle (14). FIG. 4C illustrates further tightening of the locking cap (20) and the movement of the narrow conical end (26) of the tulip portion of the screw (16) in relation to the deformable collar (18). FIG. 4D shows the transition to a poly axial head during final tightening of the locking cap (20). In this figure, the narrow conical end (26) of the tulip portion of the screw (16) has been pulled above the deformable collar (18). Transition to a poly axial head also reduces the stress that will be placed on the screw bone interface if ideal alignment of the screws or rod contouring is not achieved. Once the final tightening of the locking cap (20) is complete, the extension tabs (32) can be removed.

When the screw (10) is initially inserted into bone the base of the tulip (16) of the screw fits snuggly over the deformable collar (18), preventing angular movement of the tulip (16) relative to the shaft (11) of the screw (10). Once the desired position of the screw (10) has been achieved, the locking cap (20) is advanced to compress the rod (30) against the cradle (14) and screw head (12). The tulip portion (16) of the screw head (12) is then pulled up and over the deformable collar (18), the resistance of which is determined by the thickness of the material and the number of slots in the collar (18) to allow deformation/constriction. Some angular motion of the tulip portion of the screw (16) would be possible once compression is applied, but it is envisaged that contact between the rod and screw head, or contact between the cradle, screw head and rod would occur in such a way to lock the angle of the screw shaft relative to the rod prior to any realignment or motion of the tulip portion of the screw relative to the screw shaft. Such contact between the rod (30) and the head of the screw (12) or between the rod (30), cradle (14) and screw head (12) will ensure the alignment of the screw (10) is maintained.

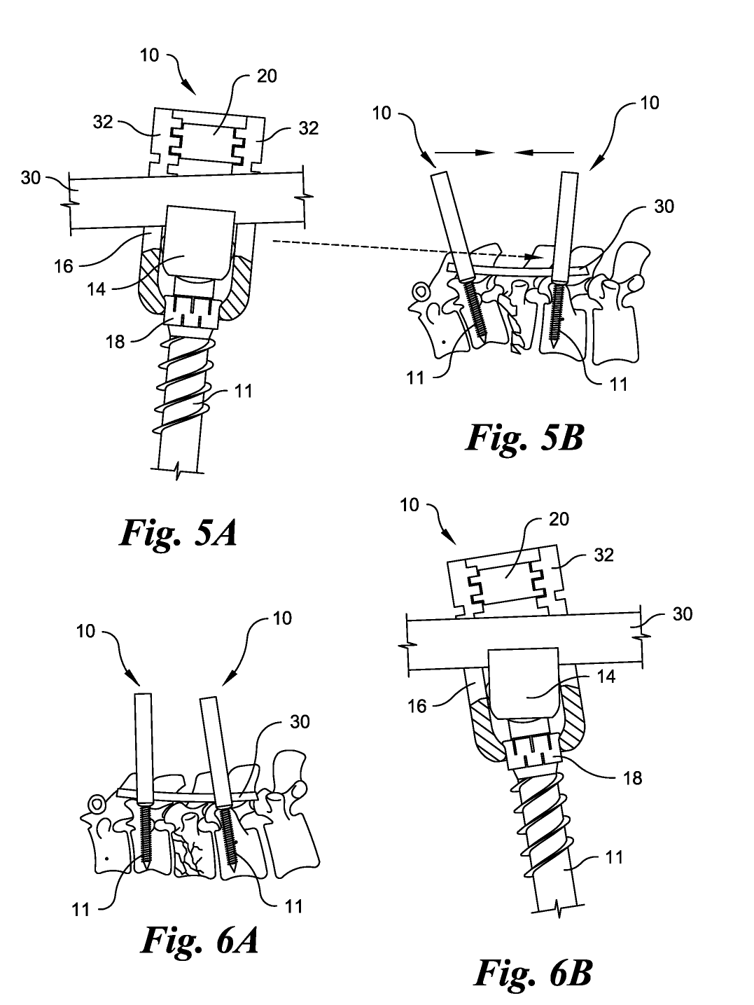

FIG. 5A illustrates a pedicle screw of the invention while FIG. 5B shows initial placement of two pedicle screws (10) of the present invention with a securing rod (30) in a trauma scenario with loss of the normal sagittal alignment of the spine. After insertion of the screws, with the screws still in the “fixed” position, the vertebrae can be manipulated to achieve the desired position with the rod (30) in place but not secured.

FIGS. 6A and 6B illustrate manipulation of the pedicle screws of the present invention (10) restoring normal alignment of the spine. The cradle (14) for the rod (30) will realign to improve the contact between the rod (30) and the screw (10).

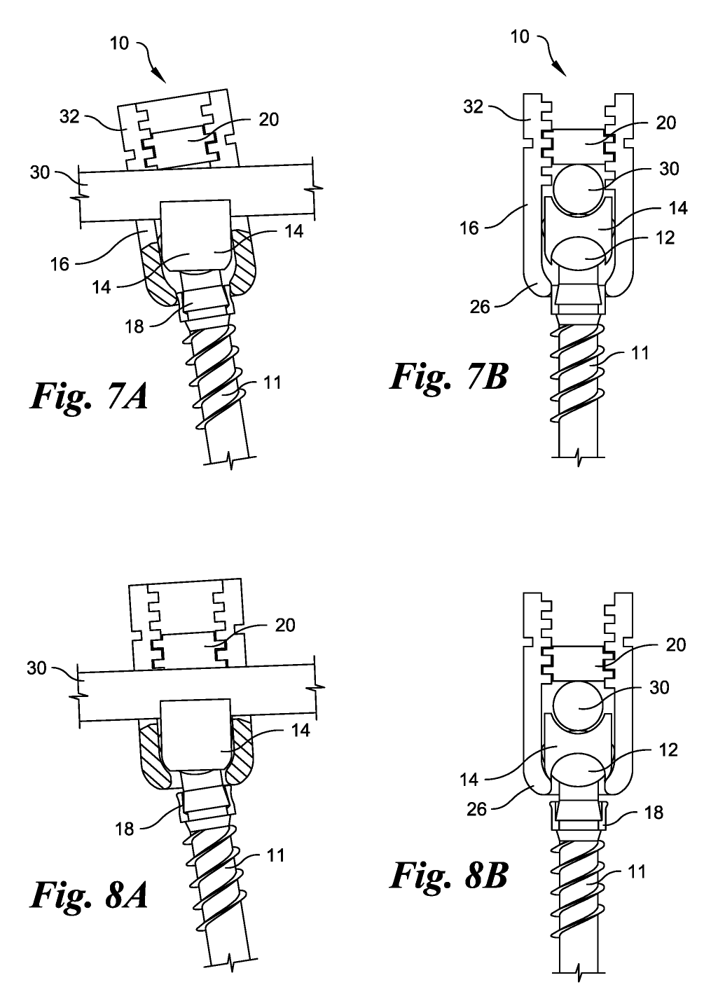

Once the desired position of the vertebrae is achieved the cap (20) can be tightened as shown in FIGS. 7A and 7B. As the cap is tightened, the rod (30) is pushed down onto the head of the screw fixing the angle of the screw shaft (11) relative to the rod (30).

FIGS. 7A and 7B depict the pedicle screw of present invention (10) with the deformable collar (18). The collar (18) is cut away to show the deformation of the proximal portion of the collar (18) as the locking cap (20) is tightened and the tulip (16) pulled up over the ridge of the collar (18) and the screw head (12).

As the cap is further tightened, the cradle (14) of the screw is drawn up along with the tulip (16) that then locks the angle of the screw shaft (11) relative to the tulip (16) and screw (10) so that the cap (20) can then be released to allow compression or distraction between the screws without loss of the position achieved and realignment or reduction of the vertebra while the screw was in its fixed configuration.

In FIGS. 8A and 8B, the pedicle screw of the present invention (10) is shown with the locking cap (20) in full contact with the rod (30). Contact and pressure transferred from the locking cap (20) through the rod (30) to the screw head (12) maintains the orientation and desired angle of the screw (10) relative to the rod (30) as the locking cap (20) is tightened.

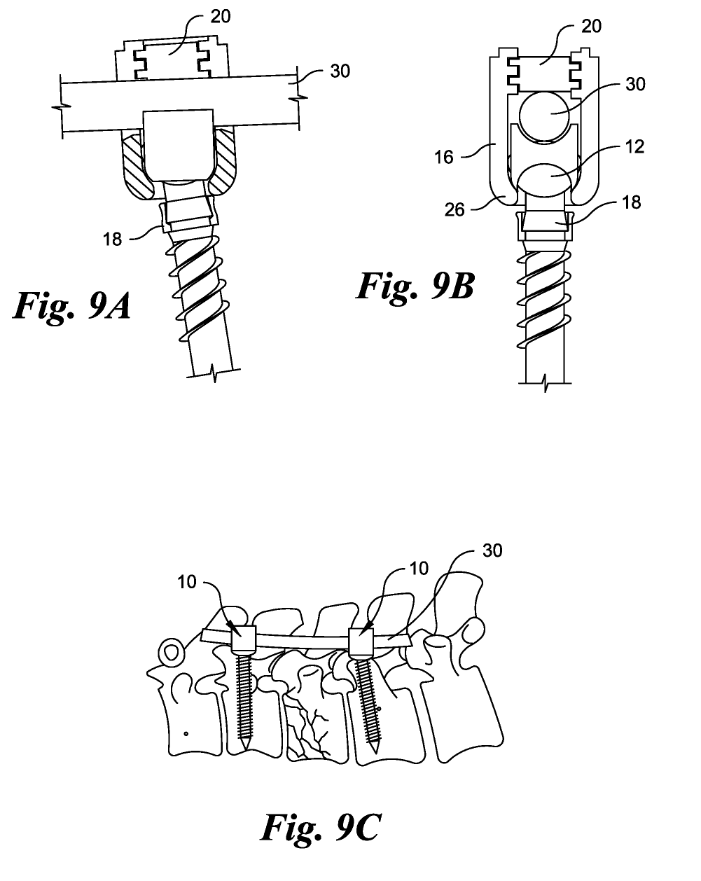

FIGS. 8A, 8B, 9A, 9B and 9C illustrate full mobility of the tulip (16) of the pedicle screw of the present invention (10). Full mobility of the tulip (16) is achieved once the base or conical portion of the tulip (26) clears the deformable collar (18). Since the upper portion of the collar (18) can be deformed, as the cap is tightened, the base of the tulip (16) is pulled over the collar (18) and the screw then becomes poly axial. However, this occurs only when the locking cap (20) is tightened pushing the rod (30) down onto the screw head (12) fixing the angle of the screw shaft (11) relative to the rod to prevent loss of reduction or realignment of the vertebrae achieved while the screw was in its fixed configuration.

FIGS. 9A and 9B illustrate the final stage of insertion, manipulation and alignment of the vertebrae where the tulip (16) aligns itself perpendicular to the rod (30) to maximize stability of the connection. Once tightened the cradle (14) locks the angle of the screw shaft (11) relative to the tulip (16) and at that point the cap (20) can be released to allow compression or distraction, where the final desired position of the screw and rod can be secured. The extension tabs (32) of the tulip (16) can then be removed.

FIG. 10 shows the identical construct before the tightening the locking cap (20) but with the use of a deformable ridge (40) below the screw head (12) and around the screw shaft (11) instead of a deformable collar.

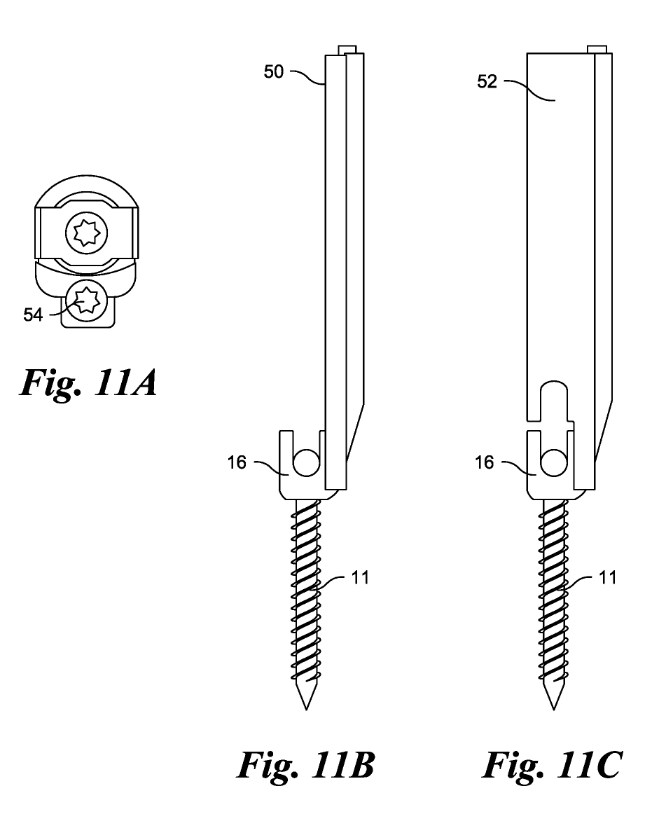

The present design is amenable to modifications of the outer configuration of the tulip to facilitate connection to tools to further manipulate the vertebral segment and reduce and hold the spine in the desired position during fixation. FIGS. 11B and 11C illustrate two options with a lateral (50) or cylindrical (52) holding/manipulating extension that attaches to the pedicle screw of the present invention. FIG. 11A is a top view of a coupling screw (54) securely attaching an extension (50) or (52) to the tulip portion of the screw.

While the present figures depict the screw with a reasonably high profile, refinement in geometry could minimize this, subject to material qualities of the ultimate metal or combination of metals used in the manufacture of this implant. This could also be overcome by counter sinking the base of the screw initially to allow translation of the tulip as outlined while minimizing the ultimate overall profile of the top of the implant relative to the posterior surface of the spine.

Furthermore, construction of the pedicle screw of the present invention can be made by any convention materials, such as, but not limited to titanium, titanium alloy and cobalt chrome.

I claim:

1. A universal mono to poly axial pedicle screw comprising a screw shaft, a screw head, a deformable member located around the screw shaft and below the screw head, a U-shaped tulip portion of the screw; wherein the tulip portion comprises a narrow conical end and an open end, wherein the narrow end surrounds the screw head and the deformable member and wherein the open end has interior opposing threads for receiving a threaded locking cap, and wherein the tulip portion of the screw further comprises a cradle with vertical sides, a first concave surface and a second concave surface opposite the first concave surface, wherein the first concave surface receives the screw head and the second concave surface receives a securing rod.

2. The universal mono to poly axial pedicle screw of claim 1 wherein the open end of the tulip portion has removable extension tabs.

3. The universal mono to poly axial pedicle screw of claim 1 wherein the cradle is cylindrical in shape.

4. The universal mono to poly axial pedicle screw of claim 1 wherein the deformable member is a deformable collar.

5. The universal mono to poly axial pedicle screw of claim 4 wherein the deformable deformable collar has one or more slots.

6. The universal mono to poly axial pedicle screw of claim 1 wherein the deformable member is a deformable ridge.

7. The universal mono to poly axial pedicle screw of claim 6 wherein the deformable ridge, engineered around the shaft of the screw below the head, prevents angulation of the U-shaped tulip prior to the application of a defined force through the tightening of the threaded locking cap down onto the rod.

8. The universal mono to poly axial pedicle screw of claim 7 wherein the deformable ridge maintains resistance to prevent loss of correction or reduction of the vertebra as the locking cap is tightened.

9. The universal mono to poly axial pedicle screw of claim 1 that screw can be used or implanted by either an open or minimally invasive approach.

10. The universal mono to poly axial pedicle screw of claim 1 wherein a deformable material prevents translation of the U-shaped tulip portion until a sufficient force is applied by tightening the threaded locking cap down onto the rod.

11. A method of securing and manipulating vertebrae of a patient spine using a universal mono to poly axial pedicle screw, wherein the universal pedicle screw comprises;
a screw shaft, a screw head, a deformable member located around the screw shaft and below the screw head, a U-shaped tulip portion of the screw; wherein the tulip portion comprises a narrow conical end and an open end, wherein the narrow end surrounds the screw head and the deformable member and wherein the open end has interior opposing threads for receiving a threaded locking cap, and wherein the tulip portion of the screw further comprises a cradle with vertical sides, a first concave surface and a second concave surface opposite the first concave surface, wherein the first concave surface receives the screw head and the second concave surface receives a securing rod;
the method comprising inserting the universal pedicle screw into a vertebra, placing the securing rod on the cradle, manipulating the vertebra, tightening the locking cap, pulling the tulip over the deformable collar, further tightening the locking cap, and orienting the tulip perpendicular to the securing rod.

12. The method of claim 11 further comprising applying compression, distraction and or rotation, without loss of angle achieved between the rod and the screw shaft, during tightening of the cap down onto the rod.

13. The method of claim 11 further comprising removing the tulip extension tabs.

14. A method of securing and manipulating vertebrae of a patient spine using a universal mono to poly axial pedicle screw, wherein the universal pedicle screw comprises;
a screw shaft, a screw head, a deformable member located around the screw shaft and below the screw head, a U-shaped tulip portion of the screw; wherein the tulip portion comprises a narrow conical end and an open end, wherein the narrow end surrounds the screw head and the deformable member and wherein the open end has interior opposing threads for receiving a threaded locking cap, and wherein the tulip portion of the screw further comprises a cradle with vertical sides, a first concave surface and a second concave surface opposite the first concave surface, wherein the first concave surface receives the screw head and the second concave surface receives a securing rod;
the method comprising inserting the universal pedicle screw into a vertebra with the screw in a fixed mono axial configuration, placing the securing rod on the cradle, manipulating the screw; and reducing or correcting positioning of the vertebra.

15. The method of claim 14, further comprising tightening the locking cap onto the rod, resulting in contact between the rod and the screw head;
fixing an angle of the screw shaft relative to the rod; and
maintaining correction or reduction of the vertebra.

16. The method of claim 14 further comprising;
tightening the locking cap;
bringing the cradle up around the screw head;
securing the rod in the cradle; and
increasing the area of contact between the screw and the rod thereby reducing point loading.

17. The method of claim 14, further comprising using a deformable material of sufficient density thereby preventing translation of the U-shaped tulip portion until a defined force is applied through the tightening of the threaded locking cap down onto the rod.

18. The method of claim 14 further comprising, tightening the locking cap, locking the position and angle of the U-shaped tulip relative to the screw shaft with the cradle; facilitating the application of compression, distraction or rotation around the rod, and maintaining or further improving reduction or correction of the spinal alignment.

19. The method of claim 14 further comprising removing the tulip extension tabs.
